(19) United States
(12) Patent Application Publication
(10) Pub. No.: US 2026/0034283 A1
(43) Pub. Date: Feb. 5, 2026

(54) Dialysate Creation From Pre-Charged Filter

(71) Applicant: Qidni Labs, Inc, Buffalo, NY (US)

(72) Inventors: Morteza Ahmadi, Kitchener (CA); Clarence Graansma, Kitchener (CA); Raman Sud, Kitchener (CA)

(21) Appl. No.: 18/788,781

(22) Filed: Jul. 30, 2024

(51) Int. Cl. A61M 1/16

(52) U.S. Cl. CPC A61M 1/1654

(57) ABSTRACT

A dialysate filter cartridge may be configured such that dialysate may be formed by passing saline solution through the dialysate filter cartridge. Once primed with saline, the dialysate cartridge may produce dialysate through recirculation through the cartridge. The dialysate filter cartridge may be configured with a higher concentration of several dialysate components such that the saline or other base liquid may absorb the components from the dialysate filter to create dialysate. The newly-formed dialysate may recirculate in the dialysis system for a period of time to equilibrate. Once the dialysate has reached a target measurement, dialysis may begin. The saline or other base liquid may be a readily available and lower cost base liquid for dialysate, and may include saline solution, distilled water, or other base liquid. For each base liquid, a corresponding dialysate filter may be configured to produce dialysate with the target components.

### BACKGROUND

Dialysis is an artificial replacement of the process of a kidney, which cleans blood or other fluids in the body. In general, dialysis uses a semipermeable membrane to remove certain harmful or toxic substances and to add or balance the concentration of other, helpful components.

Hemodialysis is a dialysis process that processes blood directly. Peritoneal dialysis is a type of dialysis that uses the body's peritoneum as a membrane to exchange with the blood.

In dialysis, a dialysate is a liquid that passes on the opposite side of a dialysis membrane from the blood or bodily fluid. During the exchange with the blood or bodily fluid, the dialysate picks up and retains the toxic and harmful components from the blood or bodily fluid, as well as transfers or regulates helpful components. In this manner, the dialysate may operate as a source for helpful components as well as a sink for harmful components.

### SUMMARY

A dialysis treatment system may use a dialysate filter for treating and cleaning dialysate. The dialysate filter may have a membrane where dialysate passes on a first side, and absorbent material is on the other side. The absorbent material may absorb urea, as well as other compounds from the dialysate. The absorbent material may supply various nutrients and compounds to the dialysate, and therefore act as a sink/source material for dialysate. The sink/source material may be a hydrogel or other protein-based material contained within a pressure-resistant housing. During the dialysis treatment, the pressure of the dialysate may be varied up and down, which increases the effectiveness of the sink/source material to clean and rejuvenate or regenerate the dialysate. The dialysis treatment system may intentionally vary the dialysis pressure during a treatment regime.

A dialysate filter cartridge may be configured such that dialysate may be formed by passing saline solution through the dialysate filter cartridge. Once primed with saline, the dialysate cartridge may produce dialysate through recirculation through the cartridge. The dialysate filter cartridge may be configured with a higher concentration of several dialysate components such that the saline or other base liquid may absorb the components from the dialysate filter to create dialysate. The newly-formed dialysate may recirculate in the dialysis system for a period of time to equilibrate. Once the dialysate has reached a target measurement, dialysis may begin. The saline or other base liquid may be a readily available and lower cost base liquid for dialysate, and may include saline solution, distilled water, or other base liquid. For each base liquid, a corresponding dialysate filter may be configured to produce dialysate with the target components.

A dialysis system may have an electrolyte management loop, which may adjust and manage electrolyte parameters for a dialysate. The electrolytes may be managed with several different electrolyte sources. By combining the different sources in different combinations, specific electrolytes may be added or adjusted to the dialysate. The electrolyte management loop may use flow-through cartridges, direct-injection, liquid or dry additive mechanisms, or combination of sources and source types to manage electrolytes. The dialysate may be managed or adjusted when the dialysate may be manufactured prior to use, as well as during use. The electrolyte management loop may adjust electrolytes during a dialysis treatment process to maintain specific electrolytes in accordance with a prescribed treatment plan for a patient.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

In the drawings,

FIG. 1 is a schematic illustration showing a dialysis system that uses dialysate filtration.

FIG. 2 is a diagram illustration showing a cut away view of a dialysate filter.

FIG. 3 is a flowchart illustration showing a method for operating a dialysis system.

FIG. 4 is a schematic illustration showing a dialysis system that manufactures dialysate from saline.

FIG. 5 is a flowchart illustration showing a method of configuring a dialysis system to make dialysate.

FIG. 6 is a schematic illustration showing a dialysis system that has adjustable parameters.

FIG. 7 is a schematic illustration showing a dialysis system that has flowable absorbent.

### DETAILED DESCRIPTION

### Overview of Recirculating Dialysate Filtering.

A dialysis system may use a recirculating system to process dialysate during a dialysis treatment. A dialysate filter cartridge may ‘clean’ or process the dialysate by absorbing urea, potassium, phosphorus, and other toxins, as well as by rejuvenating or regenerating the dialysate by providing nutrients or other beneficial components back into the dialysate. The dialysis filter cartridge may regulate several components of the dialysate, which in turn, regulate the same components of a patient's blood or other bodily fluid, such as peritoneal fluid.

Once cleaned and rejuvenated, the dialysate may return to a dialysis filter to absorb and transmit components to/from the patient's blood or other bodily fluid. Dialysate may be reused multiple times during a treatment.

The dialysate filter cartridge may have a membrane where dialysate passes on one side of the membrane, and a sink/source material resides on the other side. The filter cartridge may have a membrane in the form of many hollow fibers. The hollow fibers may be individual tubes through which the dialysate flows. On the outside of the fibers, the sink/source material may be encapsulated by a pressure-resistant housing.

The sink/source material may be, for example, a hydrogel or other protein-based material. Such material may have the consistency of a liquid or a gelatin-like material. The sink/source material may fill most or all the available volume inside the pressure-resistant housing not consumed by the membrane. In many cases, the sink/source material may come in contact with one side of the membrane so that components may traverse the membrane to be removed from or added to the dialysate. Such a movement may be through osmosis, convection, or other mechanisms.

### A Dialysis System.

The system may recirculate dialysate through the dialysis filter that processes blood or other bodily fluid. In the dialysis filter, blood or other bodily fluid may be ‘cleaned’ by transferring urea, potassium, phosphorous, and other toxins into the dialysate. Such ‘dirty’ dialysate may then be passed through the dialysate filter to remove urea and other toxins, as well as to rejuvenate the dialysate by adding various components. Such filters may remove creatinine, uric acid, beta 2 microglobulin, inulin, non-uremic toxins, urea, potassium, phosphorous, and other compounds removed from the patient's blood or other bodily fluid.

A dialysis system may operate various pumps, valves, sensors, and the like to perform a dialysis treatment. Such a machine may have a controller, such as a microprocessor, that may have instructions to prepare the machine for operation, begin a treatment, monitor performance, and end the treatment.

A recirculating dialysis system may use a fixed amount of dialysate for a treatment regimen. The amount of dialysate may be as little as 0.5 liters or less that may be recirculated. Other systems may have 1, 2, 3, 4, 5, 6 or more liters of dialysate.

The dialysate may be recirculated and reused multiple times to transfer toxic components away from the blood or other bodily fluid and into the dialysate filter. As the dialysate may be recirculated, the dialysate filter may become saturated with the components removed from the blood or other bodily fluid or otherwise become less effective at ‘cleaning’ the dialysate.

Some dialysis systems may have one, two, or more dialysate filters to clean and rejuvenate dialysate during treatment. Where two or more dialysate filters may be present, a dialysis system may operate for a period of time until a first dialysate filter may begin to become saturated with urea or other components. At such a condition, the dialysate filter may be less effective at cleaning the dialysate, and a system may switch a new dialysate filter into the flow path.

In general, dialysate filters may clean and rejuvenate dialysate at a faster or more effective rate at the beginning of their use. As the sink/source material absorbs unwanted components from the dialysate, the sink/source material may become saturated and absorb less. Therefore, switching out one filter for another may increase the cleaning and rejuvenation mechanisms during the dialysis treatment.

Multiple dialysate filter cartridges may be used in some dialysis systems. Such systems may have manifolds, valving, and other plumbing to switch out a saturated dialysate filter with a new, fresh dialysate filter. Some systems may perform a switchover automatically, while other systems may have a manual switchover process. One version of a switchover process may pause dialysis recirculation so that a dialysate filter may be switched out, while other systems may perform such switchover without pausing treatment.

Systems that contain multiple dialysate filter cartridges may have the cartridges arranged to be used one at a time, where new filter cartridges may be individually switched into the flow path. In some systems, two or more filter cartridges may be operated in parallel, where dialysate may flow in parallel through two or more filters at the same time. In still other systems, filter cartridges may be arranged to recirculate dialysate in series, where the dialysate flows through one filter cartridge, then a second filter cartridge, and so on during a dialysis treatment.

A filter may be formed in a replaceable cartridge. Some systems may use cartridge filters because the cartridges may be easily replaced, and many systems may use two, three, or more cartridges for a single treatment session. In such systems, the cartridges may be a replaceable or consumable item, while the dialysis system may be a piece of capital equipment that may be used over and over.

### The Dialysate Filter Cartridge.

The dialysate filter cartridge may have a pressure-resistant housing that contains a membrane that separates the dialysate from the sink/source material. One such design may have a multitude of hollow fiber membranes through which the dialysate flows, and the sink/source material may fill the remaining volume of the housing.

The dialysis filter cartridge may have a pressure-resistant housing that may tolerate varying pressures. In a common filter cartridge design, the operating pressure of a cartridge may be in the range plus or minus 10 psi or even 5 psi, 3 psi, 2 psi, 1 psi, or less. However, the burst strength of the cartridge may be 30 psi or more.

The sink/source material may be formed from gelatin, agar, pectin, psyllium polysaccharide, polystyrene sulfonate, poly (sodium styrene sulfonate), or other similar materials. Such material may be the bulk of the sink/source material, and various components or additives may be infused into the bulk.

The membrane and sink/source material may attempt to create an equilibrium for various components of the dialysate as the dialysate passes through the filter. For example, the sink/source material may begin a filtration process having no urea captured in the bulk.

A dialysis filter may remove the urea from the blood or bodily fluid and transfer the urea to the dialysate. The dialysate may then pass through the dialysate filter. As urea-laden dialysate passes through the dialysate filter, the lack of urea in the sink/source material may cause the urea to begin to equilibrate across the membrane through osmosis or other mechanisms, thereby traversing the membrane and being captured by the sink/source material.

In the same way, some components may be infused into the sink/source material such that a lack of the component in the dialysate may cause the component to traverse the membrane and restore or rejuvenate the dialysate with that component. For example, the sink/source material may be infused with sodium and other components. As dialysate passes through the dialysis filter, the dialysate may be kept at a predefined sodium level from the sink/source material in the dialysate filter.

In some systems, the membrane may have a pore size in excess of 50,000 Daltons. Such systems may effectively capture urea and other contaminants from the dialysate, and may add various electrolytes or other components to the dialysate. Such systems may have additional filters, such as ultra filters, that may remove leachables and other unwanted components that may have traversed the membrane from the sink/source material.

Other systems may have membranes with a pore size in the range of 20,000 Daltons. Such membranes may effectively capture the leachables and may prevent the leachables from entering the dialysate. A tradeoff between pore size of the membrane may exist, where larger pore size may have more effective transfer across the membrane, but may require an additional ultrafilter to control leachables, while the smaller pore size may remove the need for an ultrafilter but may not be as effective at passing molecules across the membrane boundary. Still other systems may have an average molecule size of the sink/source material that is 5×, 10×, 20×, or even 100× larger than the average pore size of the membrane.

The pressure-resistant housing may contain the sink/source material such that the sink/source material cannot escape or ‘leak’, especially when dialysate may be introduced at pressure. Such a configuration may force various components to traverse the membrane to process the dialysate from an unfiltered or ‘dirty’ state to a filtered or ‘cleaned’ state.

For the dialysate, an unfiltered or ‘dirty’ state may be the state of the dialysate as the dialysate exits the blood/dialysate filter, which is sometimes known as the dialysis filter. In the unfiltered state, the dialysate may contain urea, plus additional components that may be removed from the blood or other bodily fluid. These components may include potassium, sodium, phosphorus, and other components.

### Variable Pressure Operation.

During the dialysis treatment, the pressure of dialysate may affect the performance of the dialysate filter. Because the filter may be sinking and sourcing various components, the movement of components across the membrane may be enhanced by varying the pressure of the dialysate.

One theory is that varying the pressure increases the movement of components within the sink/source material. Increasing and decreasing pressure of the dialysate causes toxins and other unwanted components in the dialysate to migrate further into the bulk of the sink/source material during higher pressure cycles. This migration may increase the filter's overall capacity to store and sequester toxins during a dialysis treatment.

The theory may be based on the fact that the sink/source material may be a gelatinous material that is not a firm, solid material, but may be a thick, bulk material that may absorb toxins through various molecular migration phenomena.

During normal operation, dialysate may pass through a dialysate filter at pressures on the order of −200 mmHg to +400 mmHg or more. In some cases, the negative pressure may be as low as −300, −400, −500 mmHg or more. In some cases, the positive pressure may be as high as 500, 600, 700, or even 1000 mmHg or more.

By varying the pressure during treatment, an increased absorption rate has been demonstrated. A 25% increase in absorption has been demonstrated by varying the dialysate pressure from +400 mmHg with a 10 second dwell time to a −200 mmHg pressure with a 0 second dwell time and a 45 second transition time between maximum and minimum pressures. This ‘sawtooth’ pressure variation increased absorption by 25%. Similar, intentional pressure variations have been recorded on other tests, showing that the pressure variation dramatically improves absorption rates in a dialysate filter.

Increased absorption has been shown to occur when an average pressure of −40 to 70 mmHg may be used, but with pressure variation of plus or minus 100, 200, 300, 400, 500, 600, 700, or more mmHg. In some cases, a baseline pressure of 0 mmHg may be used, with plus or minus 300, 500, or 700 mmHg.

Pressure variation of the dialysate may be created by controlling various pumps, pressure regulating devices, and the like in the dialysate recirculation path. In some systems, such devices may be controlled by a microprocessor controller to increase and decrease the dialysate pressure as the dialysate traverses the dialysate filter.

The pressure of the dialysate when passing through the dialysate filter may be different than the pressure of the dialysate when passing through the dialysis filter. Such systems may maintain a more regulated pressure when passing through the dialysis filter, where the dialysate interacts with the blood or other bodily fluid. Such regulation may hold the dialysate pressure more constant or at a lower pressure than when the dialysate passes through the dialysate filter to clean and rejuvenate the dialysate.

In some systems, the pressure in the dialysate filter may be 5 psi or higher than the pressure in the dialysis filter, and the pressure in the dialysate filter may be intentionally controlled to increase and decrease while maintaining a relatively stable pressure in the dialysis filter. The pressure of dialysate in a dialysate filter may vary 5%, 10%, 20%, 25%, 40%, 50%, or more during a dialysis treatment. Experimental studies have shown dramatically improved absorption when dialysate pressure has been varied during a dialysis treatment.

Some systems may have a high pressure or varying pressure portion of a flow path in the area of the dialysate filter, where high pressure and varying pressure may be applied to dialysate passing through the dialysate filter. Such systems may have a more constant, more controlled, or overall lower pressure in the portion of the flow path where the dialysate passes through a dialysis filter. One example of such a system may have a high pressure recirculating pump prior to the dialysate filter, and a pressure reducing feature installed after the dialysate filter.

### Creating Dialysate Using Saline Solution.

Dialysate may be created by passing saline solution or other carrier liquid through a dialysate filter in a preliminary state. The saline solution or other carrier liquid may extract certain components from the dialysate filter to change the carrier liquid into dialysate.

In such a system, the dialysate filter may be pre-charged with various components that may be extracted from the filter to create the dialysate. Once the dialysate has been created from the dialysate filter, the dialysate filter may be in an operational state, ready to clean and rejuvenate dialysate during a dialysis treatment.

The saline or other carrier liquid may be any precursor liquid. Sterile saline solutions are often readily available and affordable, and many saline solutions may be available in different concentrations of sodium. Other precursor liquids may include sterile water, distilled water, filtered water, or even tap water in some instances. In some cases, the carrier or base liquid may be conventional 2% hypertonic saline, although saline concentrations higher than 2% may also be used.

Such systems may have dialysate filters specifically formulated for the base liquid used to manufacture dialysate. For example, a filter designed to make dialysate using sterile water may be different from a filter designed to make dialysate from 2% saline.

Many recirculating dialysate systems may use sterile dialysate so as to prevent unwanted components, including infectious agents such as viruses and other pathogens, from crossing the dialysis filter and entering a patient's blood or other bodily fluid. As such, a non-sterile precursor fluid may be filtered and sterilized prior to creating dialysate.

In a preliminary state, a dialysate filter may have an excess of various components, such as sodium, potassium, calcium, magnesium, bicarbonate, acetate, and other components. The carrier liquid may pick up such components into the carrier liquid when passing through the dialysate filter. In many cases, a dialysis system may recirculate the carrier liquid through the dialysate filter for a period of time to bring the newly-made dialysate into equilibrium. Once manufactured through this process, the dialysate may be used during a dialysis treatment by passing the dialysate through a dialysis filter to extract harmful components from the patient's blood or other bodily fluid and add or maintain helpful components to the blood or other bodily fluid.

When dialysate may be manufactured in such a technique, the dialysate filter may transform from a preliminary state to an operational state. In the preliminary state, saline or other precursor liquid may extract components from the dialysate filter to both create dialysate and to transform the dialysate filter into an operational state. In the operational state, the dialysate filter may process ‘dirty’ dialysate from the dialysis filter to clean and rejuvenate the dialysate. Over time, the dialysate filter may become increasingly saturated and become less able to absorb urea and other toxins from the dialysate.

The preliminary state of a dialysate filter may be pre-charged with various components that make up dialysate. The pre-charged amounts may be a higher level of the dialysate components than in the operational state, and the amounts of the components may be calculated to disperse into the precursor liquid and achieve an equilibrium or near-equilibrium level that may thereby create dialysate. In the operational state, the amounts of the components in the dialysate filter may maintain a desired level of the various components throughout the dialysis treatment.

Such a dialysate filter may be useful to create dialysate on demand whenever and wherever a dialysis treatment may be performed. Rather than manufacturing, storing, distributing, and using dialysate, a readily available carrier fluid, such as sterile saline, may be used instead. Such a change may dramatically lower the cost of maintaining a supply chain and various distribution channels for dialysate. Further, because sterile saline is already in widespread distribution with high availability, the cost of sterile saline may be much lower than dialysate, which may be produced in much lower volume.

### Electrolyte Management Loop.

An electrolyte management loop may contain one or more mechanisms for adjusting specific electrolytes in a dialysate. The electrolyte management loop may adjust electrolytes as part of preparation for a dialysis treatment, such as when a dialysate is manufactured prior to use. During a dialysis treatment process, the electrolytes may be monitored and adjusted to achieve a desired level of electrolytes.

The electrolyte management loop may have several independently controlled electrolyte adjustments. In a typical example, an electrolyte management loop may have an acid management loop that may add acetic acid to lower pH and may have an independent bicarbonate loop that may add sodium bicarbonate to raise pH. The acid loop may be further divided into a pure acetic acid mechanism and a mechanism that may add acetic acid and calcium chloride. Similarly, the bicarbonate loop may have a mechanism to add sodium bicarbonate and a second to add sodium bicarbonate and potassium bicarbonate.

Some systems may have lactate or other compounds that the body may convert to bicarbonate. For the purposes of this specification and claims, any reference to “bicarbonate” includes any compound, including lactate, that may be converted by the body into bicarbonate.

Such a system may allow for different variables to be independently controlled. For example, to raise or lower pH, the acetic acid or the bicarbonate mechanisms may be activated. To add calcium chloride but without changing pH, the calcium chloride and acetic acid mechanism may be activated, which may add calcium chloride but may also lower pH. To offset the pH change, the system may also activate the bicarbonate mechanism. In such a state, the system may effectively add calcium chloride to the dialysate without changing the pH.

The additive mechanisms may use some form of reservoir of additives that may be added to the dialysate in a controlled manner. One mechanism may be a flow through cartridge that may contain the additive that may be leached, absorbed, or otherwise added to the dialysate. Another mechanism may be a dry or liquid injection mechanism that may add an additive. Still other mechanisms may be used.

The additive mechanisms typically may be computer-controllable so that specific doses of additive may be added. In the example of a flow-through cartridge additive mechanism, a peristaltic pump may start, stop, and otherwise control the flow of dialysate through the cartridge. Similar controllable mechanisms may be used in injection-type additive mechanisms as well.

Throughout the drawings, schematic illustrations of peristaltic pumps are used. In many cases, the pumps used in various systems may be peristaltic pumps, however, any type of suitable pump may be used, including various positive displacement-type pumps, velocity-type pumps, impulse pumps, or any other type of pump. The illustration of peristaltic pumps should be considered a placeholder for any type of pump.

Throughout this specification, like reference numbers signify the same elements throughout the description of the figures.

When elements are referred to as being “connected” or “coupled,” the elements can be directly connected or coupled together or one or more intervening elements may also be present. In contrast, when elements are referred to as being “directly connected” or “directly coupled,” there are no intervening elements present.

In the specification and claims, references to “a processor” include multiple processors. In some cases, a process that may be performed by “a processor” may be actually performed by multiple processors on the same device or on different devices. For the purposes of this specification and claims, any reference to “a processor” shall include multiple processors, which may be on the same device or different devices, unless expressly specified otherwise.

When elements are referred to as being “connected” or “coupled,” the elements can be directly connected or coupled together or one or more intervening elements may also be present. In contrast, when elements are referred to as being “directly connected” or “directly coupled,” there are no intervening elements present.

The subject matter may be embodied as devices, systems, methods, and/or computer program products. Accordingly, some or all of the subject matter may be embodied in hardware and/or in software (including firmware, resident software, micro-code, state machines, gate arrays, etc.) Furthermore, the subject matter may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. By way of example, and not limitation, computer readable media may comprise computer storage media and communication media.

Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can accessed by an instruction execution system. Note that the computer-usable or computer-readable medium could be paper or another suitable medium upon which the program is printed, as the program can be electronically captured, via, for instance, optical scanning of the paper or other medium, then compiled, interpreted, of otherwise processed in a suitable manner, if necessary, and then stored in a computer memory.

When the subject matter is embodied in the general context of computer-executable instructions, the embodiment may comprise program modules, executed by one or more systems, computers, or other devices. Generally, program modules include routines, programs, objects, components, data structures, etc. that perform particular tasks or implement particular abstract data types. Typically, the functionality of the program modules may be combined or distributed as desired in various embodiments.

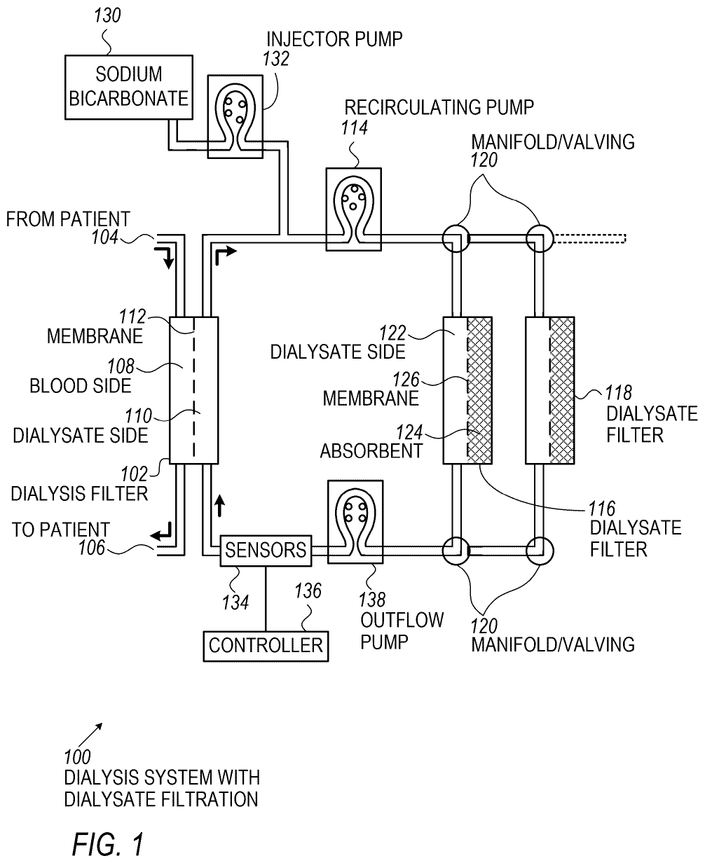

FIG. 1 is a diagram or schematic illustration of an embodiment 100 showing a dialysis system with dialysate filtration. Embodiment 100 is a schematic example of the various components that may be used to perform dialysis. During the dialysis treatment, the dialysate may remove components from the patient's blood, such as urea. The dialysate may then be ‘cleaned’ by a dialysate filter and rejuvenated with various components that may be passed back to the blood.

The recirculation process may ‘clean’ the blood by transferring unwanted components from the blood to the dialysate, then the process may ‘clean’ the dialysate by transferring those unwanted components to an absorbent material in the dialysate filter. In the same way, the absorbent material may have some components that may be added into the dialysate, which in turn may add those components to the patient's blood.

A dialysis filter 102 may receive blood or other fluid from a patient 104, may pass the blood or other fluid through the dialysis filter 102, then return the blood or other fluid to the patient 106. The dialysis filter 102 may be conceptually represented as having a blood side 108 and a dialysate side 110, which are separated by a membrane 112.

It should be noted that throughout this specification and claims, unless specifically called out otherwise, the term “blood” being treated in a dialysis system may include blood or any bodily fluid that may be treated in a dialysis system. In many instances, dialysis may be used to treat peritoneal fluids, blood, or any other bodily fluids. As such, the term “blood,” as used in this specification and claims, shall be a shorthand notation for any bodily fluid of any patient being treated. The patient may be human or any type of animal.

The membrane 112 may be a semi-permeable membrane that may allow certain components, such as urea, to be extracted from the blood through osmosis, mechanical filtering, or other physical phenomena. The membrane 112 may have many different mechanical configurations, and typical versions may attempt to maximize the surface area of the membrane to enhance the transfer across the membrane.

A hollow fiber membrane may be a version of the membrane 112 that has very large surface area and very effective transfer across the membrane 112. In a hollow fiber membrane, either the blood or the dialysate may pass through the center of a hollow fiber. The hollow fiber or tube conducts the first liquid through the filter, while the second liquid passes on the outside of the hollow fiber or tube. Such filters often have hollow fibers sized at or below a millimeter, and a single filter may have hundreds or even thousands of the hollow fiber membranes.

After passing through the dialysis filter 102, the dialysate may be ‘dirty’, having received urea and other components from the blood. The dialysate may be recirculated by a recirculation pump 114, then pass through a dialysate filter 116.

A dialysate filter 116 may have a dialysate side 122 and an absorbent side 124, separated by a membrane 126. In general, the absorbent side 124 may contain an absorbent that may be a hydrogel or other material. As such, the absorbent may be a gelatinous material. Sometimes, the material may be viscous, solid, semi-solid, or have other physical characteristics.

Throughout this specification and claims, the term “absorbent” may be used in place of “sink/source material.” Absorbent may be a shorthand notation that may include material that operates as sink, where the material accepts or absorbs another material, or as a source, where the material provides, distributes, or transmits another material. During a dialysis treatment, the “absorbent” may, indeed, absorb urea and other ‘dirty’ components in the dialysate. At the same time, the “absorbent” may provide, regulate, or otherwise maintain a desired level of other components. Such a process may be referred to as ‘rejuvenating’ the dialysate. The term “absorbent,” as used herein, refers to material that performs both the sinking or removal of certain components and the sourcing or addition of other components, typically to the dialysate.

The sink/source material or absorbent 124 may be a material that may be biocompatible with the patient. Even though the absorbent 124 may not have direct contact with the patient or the patient's bodily fluids, by making the absorbent 124 out of a biocompatible and hemocompatible material, any possible leakage, membrane failure, or other abnormality may not inadvertently cause a problem with the patient.

The dialysate filter 116 may have an absorbent that may not flow or recirculate. As such, the absorbent may absorb components from the dialysate and supply other components to the dialysate during a dialysis treatment. At some point, the absorbent may become saturated or otherwise less effective, as the concentration gradient between the dialysate and the absorbent decreases.

Multiple dialysate filters may be used in some systems. Such systems may flow dialysate through a first dialysate filter 116 until that filter becomes saturated or at least less capable of absorbing, at which point, a second dialysate filter 118 may be switched into the flow path. A manifold or various valving 120 may be configured to switch out one dialysate filter 116 and switch in a second dialysate filter 118. Some systems may have two, three, four, five, or more filters available to switch in and out during a dialysis treatment.

The system 102 may illustrate two or more dialysate filters 116 and 118 with the manifold and valving 120. Such a system may automatically change from one filter to another during treatment. Other systems may have a manual changeover from one filter to another. Such a manual system may determine that the current filter may be saturated, then may pause the system, send an alarm, or otherwise indicate that a person may remove the existing filter and replace the old filter with a new one.

Some systems may switch between dialysate filters without pausing or stopping a dialysis treatment. Other systems may pause the dialysis treatment and the dialysis recirculation, then reconfigure the valves to cause the flow through a new filter, then resume the treatment. Such systems may coordinate the dialysate recirculation pump 114 with a blood recirculation pump, which is not shown in the diagram, to pause and resume the treatment. Still other systems may pause the dialysate recirculation for a short period to switch to a new filter, all the while maintaining a flow of blood from the patient.

An outflow pump 138 may be used in conjunction with the recirculating pump 114 to control and vary the pressure of dialysate as the dialysate passes through one or more of the dialysate filters 116 and 118. In order to increase pressure of dialysate inside a dialysate filter, the recirculating pump 114 may be operated at a higher speed while the outflow pump 138 may be operated at a slower speed. A negative pressure may be created by running the outflow pump 138 at a higher speed while operating the recirculating pump 114 at a lower speed.

Using two peristaltic pumps as the recirculating pump 114 and outflow pump 138, a wide range of fluid pressure may be introduced to the dialysate as it passes through the dialysate filters 116 and 118. The pressure variation may be controlled in the region of the filters, but may be held at a more constant pressure when the dialysate passes through the dialysate filter 102. In such a manner, the dialysate may operate in two different pressure ranges within the same circulation loop.

A set of sensors 134 may be monitored using a controller 136 to manage a dialysis treatment. The controller 136 may turn on and off the various pumps, valving, sensors, and other components of the dialysis system during startup, setup, priming, operation, and tear down and clean up. The controller 136 may collect data about the treatment, note any problems, send alerts regarding problems, and provide a user interface through which a patient or caregiver may operate the system.

Some systems may have an injector pump 132 through which additional materials, such as sodium bicarbonate 130 may be added to the dialysate. Sodium bicarbonate 130 may be one example of a component that may be added to the dialysate, and other systems may have other similar components.

In the example, the sodium bicarbonate 130 may be a liquid form having a predetermined dilution of sodium bicarbonate. A sensor may determine that the acidity level, conductivity, or other parameter of the dialysate may be out of specification. The controller 136 may receive an input from one or more sensors 134 and in response, may cause an injector pump 132 to inject a material into the dialysate. In the example, such material may be sodium bicarbonate. The controller 136 may monitor the sensors 134 to determine if the parameter is brought back into specification. In the case where a parameter may not be brought back into specification, an alarm may be indicated. In some cases, such alarms may merely warn a caregiver or operator of the machine, while in other cases, some such alarms may cause the system to pause or stop operation.

One sensor 134 may be a pH sensor. Some systems may us a pH sensor to determine if and when to cause sodium bicarbonate 130 to be injected using the injector pump 132. In one example of such a system, sodium bicarbonate may be added to maintain a pH between 6.5 and 7.8. Such systems may include an acid in the dialysate filter absorbent material, such as acetic acid, citric acid, lactic acid, or another acid that may maintain a pH of less than 4.5 prior to adding any sodium bicarbonate. Such a system may begin with a low pH, then use the sodium bicarbonate to regulate and control the pH prior to and during the dialysis treatment.

Another sensor may be a conductivity sensor. A conductivity sensor may indicate the cleanliness or completeness of filtration for a dialysate, and a conductivity sensor may be located downstream from the dialysate filter. A conductivity between 12.0 mS and 16.0 mS has been shown to be an effective range for properly filtered dialysate. When the conductivity may exceed this range, a system may determine that the current dialysate filter may be saturated and another filter may be inserted into the flow path.

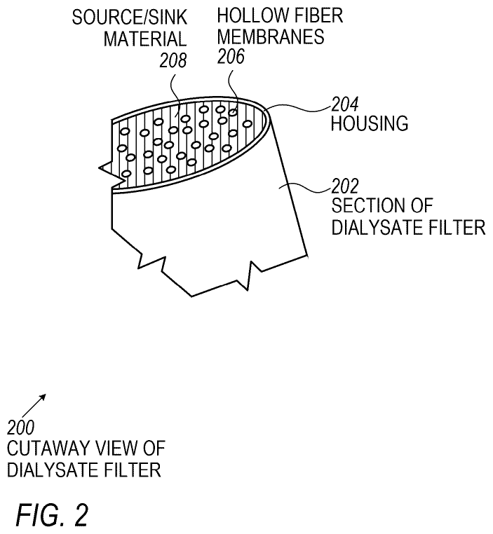

FIG. 2 is a diagram illustration of an embodiment 200 showing a cutaway view of a dialysate filter. Embodiment 200 is not to scale, and is drawn to highlight several components and features of an example dialysate filter.

In the section 202 of a dialysate filter, a housing 204 holds the internal materials, which may include a plurality of hollow fiber membranes 206 and the sink/source material 208, otherwise known as the absorbent. The operation of the filter 202 may involve passing dialysate through the hollow fiber membranes 206.

The dialysate in the hollow fiber membranes 206 may transfer components into the sink/source material 208 and, in some cases, receive components from the sink/source material 208.

The sink/source material 208 may be a gelatinous material, made from any of several types of naturally occurring or artificial polymer materials. Examples of the base component of the sink/source material 208 may include gelatin, agar, pectin, cellulose, psyllium polysaccharide, polystyrene sulfonate, poly (sodium styrene sulfonate), chitosan, or other similar materials. The bulk material may be cross-linked in many instances. Some materials may be at least partially cross-linked, while other materials may be highly or even nearly fully cross-linked. In some cases, the cross-linking may occur in situ, after the base material may be added to or encapsulated in a housing 204.

In general, the base material for the sink/source material 208 may have a very high surface area to bind urea and other components removed from the dialysate, as well as the capability to be mixed with or charged with other components that may be added to the dialysate or otherwise regulated within the dialysate. In many cases, the base material may be gelatinous or have high viscosity.

One explanation for the absorption mechanism may be diffusive and/or convective transfer across the membrane and through the sink/source material 208. In a diffusive transfer, a concentration gradient between the dialysate and the sink/source material 208 may exist, causing transfer across the membrane. Once a component has crossed the membrane, a concentration gradient may exist within the sink/source material 208, causing further diffusion within the sink/source material 208. Such a mechanism may explain how components, such as urea, may be removed from the dialysate and stored in the sink/source material 208. Such a mechanism may also explain how higher concentrations of various components, such as the electrolytes of sodium, potassium, calcium, magnesium, chlorides, bicarbonate, as well as the concentration of an acid such as acetic acid, citric acid or lactic acid may be transferred from the sink/source material 208 to the dialysate.

A housing 204 may be designed to be pressurized. In many situations, the absorption from the dialysate may be improved by operating the filter 202 at pressures above 2 psi, sometimes in the range of 5 to 10psi. However, by varying the pressure 10% or more, sometimes 25%, 50%, or even approaching 100%, the absorption of urea and other components may improve dramatically.

The sink/source material 208 may comprise a base material, which may be the bulk of the volume and weight, and a group of infusions or additives, which may be a smaller portion of the volume and weight. The base sink/source material may have a molecular weight greater than 50,000 daltons, and some versions have molecular weights greater than 60,000, 70,000, 80,000, 100,000 or even 500,000, 700,000, 1,000,000 Da, or more. The high molecular weight may prevent the sink/source material from traversing the dialysate membrane.

The base material may comprise a large amount of the total weight of the sink/source material. In many cases, the base material may comprise 95% of the total weight of the sink/source material, although some sink/source materials may have 90%, 80%, or even 60% of the total weight comprised of the base material. Some systems may have base material comprising 97%, 98%, or higher of the base material as part of the sink/source material 208.

Many dialysate systems may have a recirculation pumping system capable of increasing and decreasing dialysate pressure. In some systems, the dialysate pressure may be increased through the dialysate filter, while the pressure of the dialysate in the dialysis filter may be much lower and held steadier, or at least without as large pressure variations. Such systems may have a recirculating pump upstream from the dialysate filter, then a pressure regulator or orifice after the dialysate filter. Such a configuration may increase dialysate pressure within the dialysate filter while having a lower pressure when the dialysate passes through the dialysis filter.

When the sink/source material 208 may be a hydrogel, the hydrogel may absorb water and swell. The absorption process may transfer components across the membrane boundary to absorb the components in the hydrogel's microporous matrix structure. When absorbed, the components may then be prevented from releasing back into the dialysate.

When the sink/source material 208 may have a natural tendency to swell, the housing 204 may be sealed, capped, or otherwise closed to prevent the sink/source material 208 from expanding or leaking out of the housing 204. In many such designs, the housing 204 may have one or more filling ports through which the sink/source material 208 may be added, then a pressure-resistant seal or cap may be applied. Such designs may experience an increased internal pressure as water may be absorbed into the sink/source material 208 and swelling or expansion may occur. In many such designs, the housing 204 may be a fixed volume that resists the swelling or expansion of the sink/source material 208.

The hollow fiber membranes 206 may prevent the sink/source material 208 from passing into the dialysate, yet various electrolytes contained in the dialysate may pass from the dialysate into the sink/source material 208. The membranes may control leaching of polymers and monomers of the sink/source material 208 into the dialysate, which in turn may prevent those polymers and monomers from reaching a patient's blood or other bodily fluid. The hollow fiber membranes 206 may be manufactured from polysulfone, polyether sulfone, or other materials or combinations of materials. In some cases, the hollow fiber membranes 206 may be similar to or identical to hollow fiber membranes used in dialysis filters.

In many cases, the sink/source material 208 may contain various components designed to pass from the sink/source material 208 to the dialysate, thereby keeping the dialysate at an equilibrium concentration of those components and rejuvenating the dialysate in some circumstances.

A filter cartridge with hollow fiber membranes 206 may contain at least 1 square meter of diffusive area and 150 ml of sink/source material 208. Some cartridges may contain 1.5 square meters of surface area, while others may contain 2 square meters, 2.5 square meters, 3 square meters, or more of surface area. Some cartridges may contain 200 ml of sink/source material, while others may contain 300 ml, 400 ml, 500 ml, 750 ml, or even more of sink/source material.

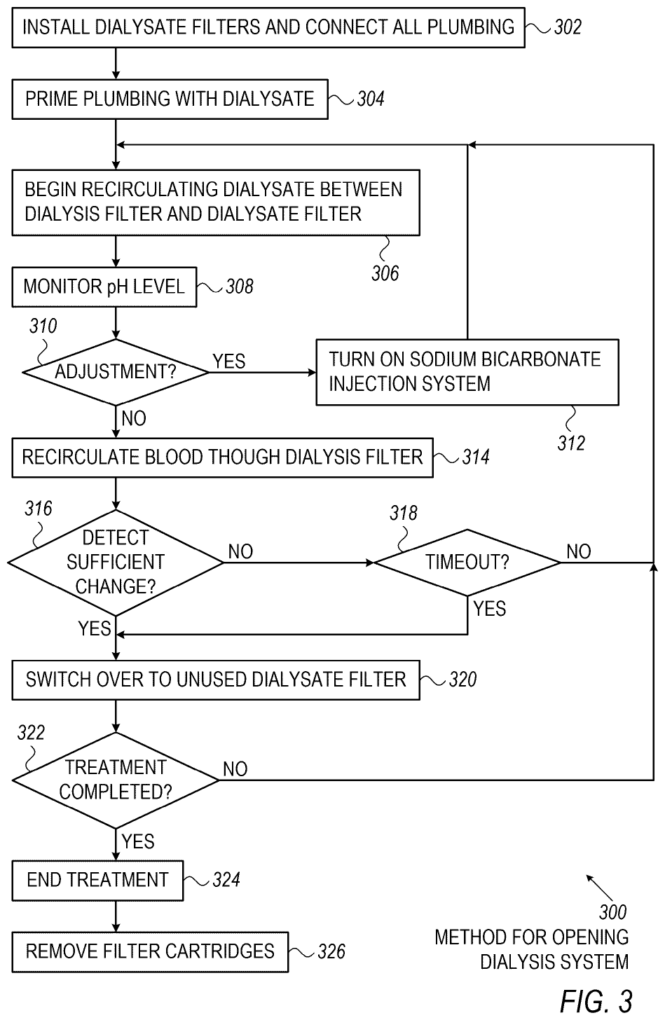

FIG. 3 is a flowchart illustration of an embodiment 300 showing a method performed by a dialysis system. The method of embodiment 300 may be one simplified example of how a dialysis system may operate to extract unwanted components from blood or other bodily fluid using a recirculating dialysate system. As dialysate filters become saturated or otherwise lose performance, additional filters may be swapped into the recirculating circuit.

Other embodiments may use different sequencing, additional or fewer steps, and different nomenclature or terminology to accomplish similar functions. In some embodiments, various operations or set of operations may be performed in parallel with other operations, either in a synchronous or asynchronous manner. The steps selected here were chosen to illustrate some principles of operations in a simplified form.

In block 302, dialysate filters may be installed and connected to any plumbing. Additionally, dialysis filters, monitors, tubing, and any other step to set up and configure the dialysis system may be performed. The set up sequence may include priming the plumbing with dialysate in block 304, which may include removing air from the tubing or other plumbing.

Dialysate may be recirculated between the dialysis filter and the dialysate filter in block 306. During this step, the system may begin functioning to ensure proper working order, as well as to monitor the pH level in block 308. If the pH level needs adjustment in block 310, the sodium bicarbonate injection system may be activated in block 312. The system may continue to recirculate in block 306 while monitoring continues in block 308 until the pH level in block 310 no longer needs adjustment.

Once the pH level of the dialysate has been established and is stable, blood may be introduced into the dialysis filter in block 314. During this stage, impurities, such as urea, may be removed from blood in the dialysis filter, transferring the impurities to the dialysate. The dialysate may circulate through a dialysate filter, which may remove and capture the components extracted from the blood, such as urea. Additionally, the dialysate may transfer certain components to the blood in the dialysis filter, and those components may be refreshed or rejuvenated by the dialysate filter.

During the recirculation process, sensors may monitor the dialysate. If there has been sufficient change in the capacity or capability of the dialysate to perform its function in block 316, the system may switch over to a new dialysate filter in block 320. In some systems, the switch over process may be an automated changeover controlled and executed by a computer controller. In other systems, some part of the switch over may be a manual process where a human removes the used filter and inserts a new one.

Some systems may cycle dialysate through a dialysate filter for a predetermined maximum period of time. Even if the sensors have not detected a deficiency in the dialysate in block 316, a timeout timer in block 318 may cause the system to change over to a new filter in block 320. While the sensors detect that the dialysate maintains within predefined limits in block 316 and the timeout of block 318 has not been exceeded, the process may continue recirculating dialysate and process blood in block 306.

In block 322, if treatment has been completed, the process will end in block 324 and any used cartridges may be removed in block 326.

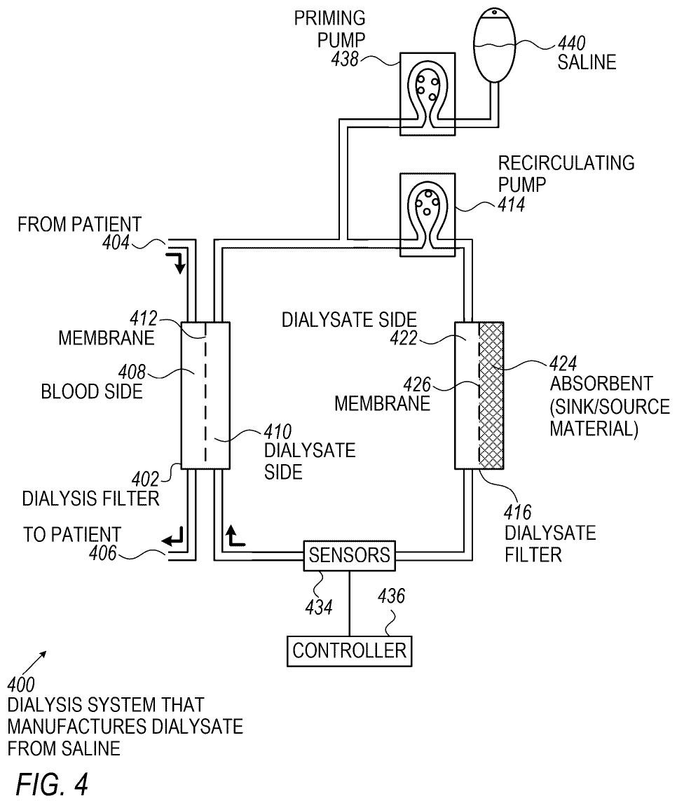

FIG. 4 is a schematic illustration of an embodiment 400 showing a dialysis system that may create or manufacture dialysate from saline or other base liquid. The diagram illustrates a system that may pass saline or other base liquid, such as distilled water, through a pre-configured dialysate filter. The base liquid may receive various components from the dialysate filter, thereby converting the base liquid into dialysate for a dialysis treatment. During the course of a treatment, the dialysate filter may maintain the appropriate levels of the dialysate components, and at the same time, may remove components extracted or absorbed from the patient's blood.

The system shows a dialysate filter that begins in a preliminary state. In the preliminary state, the dialysate filter may be charged with excess levels of various dialysate components. The excess levels may be selected so that when saline or other base liquid passes through the dialysate filter in the preliminary state, those components are passed to the dialysate through osmosis or other phenomena to reach an equilibrium or near-equilibrium concentration. At such concentration, some components may be transferred to the patient's blood, and the reserve capacity in the dialysate filter may rejuvenate, restore, or otherwise maintain the concentration in the dialysate during the dialysis procedure.

A dialysis filter 402 may receive blood or other bodily fluid from a patient 404, process the blood or other bodily fluid, and return the blood or other bodily fluid to the patient 406. Schematically, the dialysis filter 402 may have a blood side 408 and a dialysate side 410, separated by a membrane 412. The membrane 412 may be any configuration of a mechanical, semi-permeable membrane between the blood side 408 and dialysate side 410. In many instances, the membrane 412 may be a set of small hollow fibers through which the blood may pass, and the filter may have inlet and outlet ports through which the dialysate may pass on the opposite side of the hollow fiber.

A recirculating pump 414 may recirculate dialysate between the dialysis filter 402 and a dialysate filter 416. The dialysate filter 416 may have a dialysate side 422 and an absorbent 424, separated by a membrane 426. The absorbent 424 may be a gel, liquid, solid, or other material that may absorb certain components, such as urea, and thereby act as a sink for those components. The absorbent 424 may also be infused or otherwise charged with various components that may be transferred into the dialysate, and thereby act as a source for those components.

In many cases, the dialysate filter 416 may have a constrained physical volume. For example, a dialysate filter 416 containing hollow fiber membranes may be filled with an absorbent 424. The filter may have a port or set of ports that may allow the filter to be filled with absorbent, then capped such that much of the air in the filter may be removed. The capped ports may prevent the absorbent from flowing or leaking out of the port when the dialysate filter 416 may be pressurized with dialysate.

Various sensors 434 may monitor the dialysate and may feed inputs to a controller 436. The controller 436 may configure the system prior to a dialysis treatment, may monitor the ongoing performance of the system during a dialysis treatment, and may perform various processes to shut down the system after dialysis has completed. The controller 436 may have various user and network interfaces to communicate the system status, send alarms, report data during and after treatment, and other functions.

A priming pump 438 may inject saline 440 or other base liquid into the system prior to treatment. The saline 440 may pass through the dialysate filter in a preliminary configuration, and the saline 440 may extract various components from the absorbent 424 to create or manufacture dialysate. The priming pump 438 may introduce liquid throughout the plumbing and tubing of the system, including the dialysis filter 402 and dialysate filter 416, thereby preparing the system for a dialysis treatment.

A base liquid, such as saline 440, may be passed through the dialysate filter 422 to create a dialysate. As various components may be extracted from the absorbent into the base liquid, the dialysis filter 416 may change into an operational state. The change from a preliminary state to an operational state may involve lowering the concentration of the dialysate components from the absorbent 424 by extracting those components from the absorbent 424. In the operational state, the dialysate filter may have a changed concentration for those components, and the concentration of those components may be lower than the initial concentration. However, the lower concentration may be a desired concentration for maintaining the concentration of those components during a dialysis treatment. During a dialysis treatment, certain components in the dialysate may be extracted from the dialysate into the patient's blood or other bodily fluid, and the absorbent 424 in the dialysate filter may restore the component, at least partially, in the dialysate during the treatment.

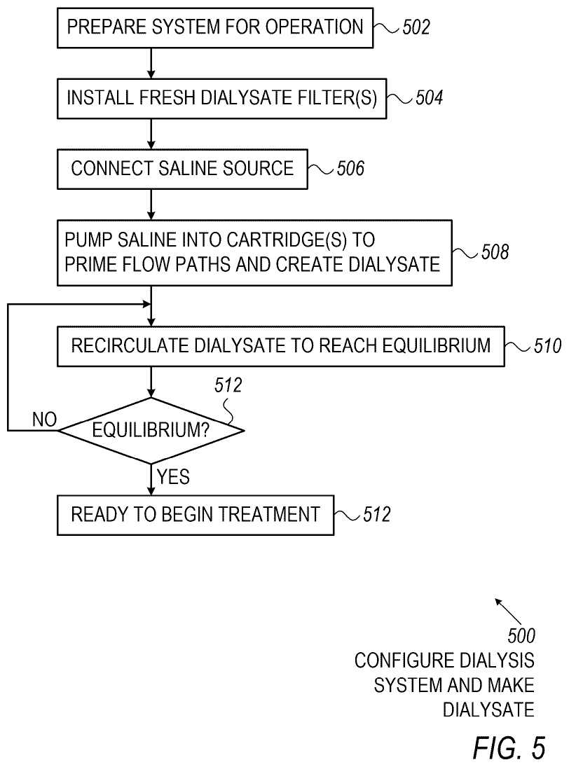

FIG. 5 is a flowchart illustration of an embodiment 500 showing a method to prime and prepare a dialysis system that creates its own dialysate. The method of embodiment 500 may be one simplified example of how a dialysis system may receive a base liquid, such as saline, and create or manufacture dialysate by extracting dialysate components from a dialysate filter. The dialysate filter may be pre-charged with dialysate components sufficient to create a dialysate by transferring excess levels from the filter to the base liquid. During dialysis treatment, the same filter may filter unwanted components from dialysate while rejuvenating or adding other components to the dialysate.

Other embodiments may use different sequencing, additional or fewer steps, and different nomenclature or terminology to accomplish similar functions. In some embodiments, various operations or sets of operations may be performed in parallel with other operations, either in a synchronous or asynchronous manner. The steps selected here were chosen to illustrate some principles of operations in a simplified form.

In block 502, the system may be prepared for operation. As many systems may have disposable tubing, the tubing set may be installed as well as other preparatory actions may take place.

Fresh dialysate filters may be installed in block 504. Some systems may use one filter per treatment, while other systems may use two, three, four, or more filters. The filters may be installed and connected in block 504.

A saline or other base liquid may be connected in block 506, and the base liquid may be pumped through the cartridge(s) to prime the plumbing system and to create dialysate. The dialysate may be recirculated in block 508 until equilibrium or near-equilibrium may be reached in block 510. During the recirculation phase of block 510, the process may return to block 508. Once the dialysate has reached equilibrium or near-equilibrium in block 510, the system may begin treatment in block 512.

Some systems may have multiple cartridges. In such systems, the priming operation of block 508 may pass the base liquid, which may be saline, through just one or sometimes two or more filters to create the dialysate. In systems where one filter may be used to create the dialysate, that first filter may have different levels of components such that after the dialysate reaches equilibrium or near-equilibrium levels, the filter may have the appropriate concentrations for dialysis operation.

In other systems with multiple filters, sometimes all of the filters may have a preliminary state that may be super-charged with dialysate components. During the conversion of the saline or other base liquid to dialysate, such systems may pass the base liquid through each of the dialysate filters. Sometimes, such operation may cycle base liquid through each filter in sequence or series, such as recirculating through one filter for a period of time, then changing to a second filter and so on. Some systems may recirculate the base liquid through two or more, and sometimes all the filters in parallel.

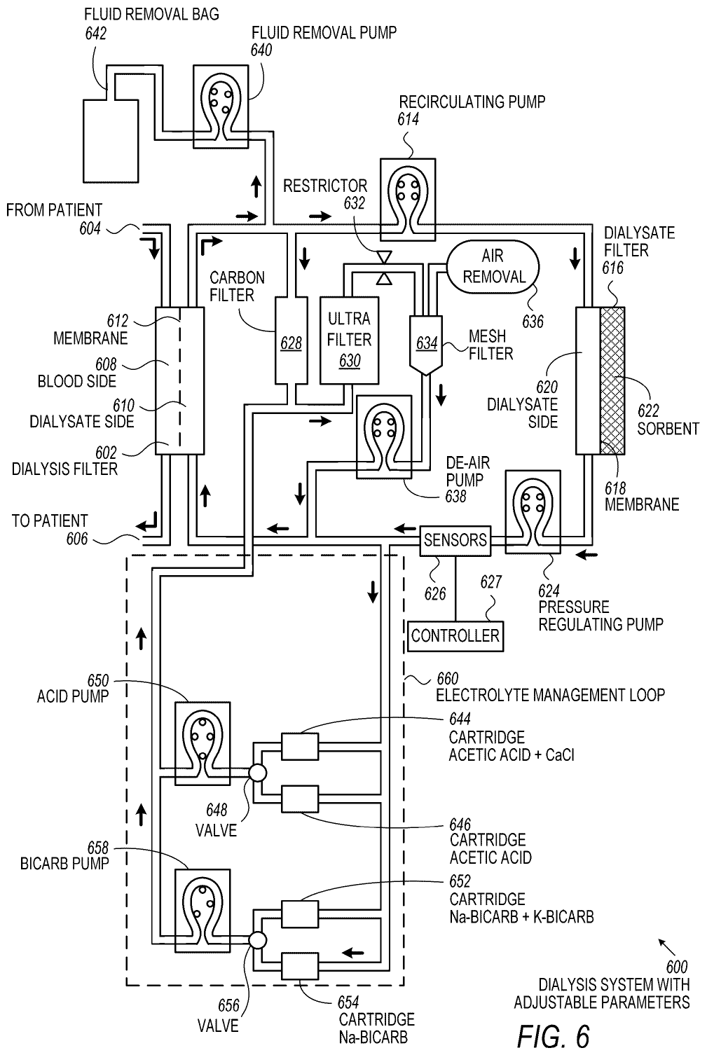

FIG. 6 is a schematic diagram illustration of an embodiment 600 showing a recirculating dialysis system having an electrolyte management system. Embodiment 600 is one illustration of how an electrolyte management system may be deployed in a dialysis system, and the example also illustrates a fluid management system that includes air removal.

A dialysis filter 602 processes fluid from a patient. The fluid may enter at an input 604 and may exit at an output 606. The fluid may be a parenteral fluid, blood, or other fluid. The dialysis filter 602 may have a blood side 608 and a dialysate side 610, separated by a membrane 612.

The dialysate may be a fluid that may capture certain components from the bodily fluid, as well as recharge or rejuvenate other components into the blood. A recirculating pump 614 may send the dialysate through a dialysate filter 616, which may have a membrane 618. The membrane 618 may have a dialysate side 620 and a sorbent side 622, and the membrane 618 may cause unwanted components to be transferred into the sorbent, such components including urea and other components. The sorbent may also recharge or rejuvenate the dialysate with components that may be transferred into the patient at the dialysis filter 602.

A pressure regulating pump 624 may regulate or manage the dialysate pressure by regulating the difference between the speed at which the recirculating pump 614 operates and the speed of the pressure regulating pump 624.

An air removal system may include a carbon filter 628 and an ultra filter 630, which may receive dialysate from the dialysis filter 602 in parallel to dialysate that may travel to the dialysate filter 616. A restrictor 632 may increase the pressure of the dialysate, which may be released in a mesh filter 634. The pressure release coupled with the mesh filter 634 may cause air entrained in the dialysate to come out and be removed in an air removal device 636. A de-air pump 638 may regulate the pressure and amount of dialysate that may be processed by the air removal system.

The ultrafilter 630 may have an average pore size in the range of 5,000, 10,000, 20,000, 30,000, or 40,000 Daltons. Such a pore size may capture leachable materials that may have entered the dialysate from the sorbent 622, as well as other contaminants in the dialysate. Such a pore size may be designed to remove bacteria as well as endotoxins from the dialysate.

The ultrafilter 630 may be sized to operate throughout a standard dialysis treatment. During the treatment, the ultrafilter 630 may capture various components, such as endotoxins, leachables, and the like, which may cause the flow rate to drop somewhat during the course of treatment. Many systems may size the ultrafilter sufficiently large so that the pressure increase or drop in flow rate may be within a tolerance such that a successful dialysis treatment may be completed. Once completed, the ultrafilter 630 may be replaced prior to performing another treatment.

A fluid removal pump 640 and a fluid removal bag 642 may be used to remove dialysate from the system when the system has completed a treatment. The fluid removal system may remove some, most, or all of the dialysate from the system to aid in replacing the various tubing and other consumable items of the dialysis system between patients.

An electrolyte management system 660 may include an acid path and a bicarbonate path. An acid path may include one, two, or more cartridges 644 and 646, which may be added or removed from the flow path by a valve 648. An acid path pump 650 may cause the dialysate to pass through the acid path to adjust the acid electrolytes. Similarly, a bicarbonate path may include one, two, or more cartridges 652 and 654 connecting with a valve 656 and driven by a bicarbonate pump 658. For the purposes of this specification and claims, the terms “acid pump” and “bicarbonate pump” may be specific instances of a generic term “electrolyte pump.” The electrolyte pump may be any type of pump, but typically a peristaltic pump, which may be controllable by a computerized controller to add specific amounts of electrolyte to the dialysate.

The electrolyte management system 660 as illustrated may include flow-through cartridges that may add various electrolytes into the dialysate. As the dialysate passes through a cartridge, one or more electrolytes may be added to the dialysate through absorption or other physical transfer mechanisms. Other additive mechanisms may include infusion mechanisms, which may add liquid or solid material directly into the dialysate.

The electrolyte management system 660 may use offsetting electrolytes to adjust various parameters. For example, an acetic acid cartridge may also include calcium chloride. To offset the pH change introduced by the acetic acid, an electrolyte management system 660 may, at the same time, introduce bicarbonate, thereby keeping the pH constant while adding calcium chloride. Using such a system, various electrolytes may be independently added in a controlled manner.

In the example of the electrolyte management system 660, electrolyte additive mechanisms may add acetic acid, acetic acid plus calcium carbonate, sodium bicarbonate, and sodium bicarbonate plus potassium bicarbonate. Such a configuration may be able to add and adjust pH, sodium, calcium, and potassium electrolytes in a dialysate.

The electrolyte management system 660 may be used to configure a dialysate prior to use. For example, a dialysate may be created by passing saline or other base liquid through one or more unused dialysate cartridges, then the specific electrolyte composition may be changed or configured using the electrolyte management system 660.

Such a system may allow a standardized set of dialysate filters to generate a ‘generic’ dialysate, then have the electrolyte management system 660 adjust the dialysate for a particular patient's prescribed treatment. Some patients may, for example, use a dialysate with higher or lower levels of sodium, potassium, or other electrolytes, and using the electrolyte management system 660, the dialysate may be specifically configured for those patients.

In some use cases, the electrolyte management system 660 may adjust electrolytes during a dialysis treatment. In such cases, a set of sensors 626 and a controller 627 may identify an electrolyte or set of electrolytes where the levels of those electrolytes may have fallen. In response, the electrolyte management system 660 may cause those electrolytes to be added, recharged, or otherwise rejuvenated to the dialysate. Such systems may hold the desired electrolyte levels constant through a dialysis treatment, however, some systems may adjust one or more electrolytes to ramp up or down throughout the treatment schedule.

The controller 627 may manage the overall setup, configuration, operation, and tear down of the system 600. During the setup phase, the controller may cause the various pumps, valves, and other components to setup the system with dialysate, purge excess air, recirculate the dialysate and adjust the dialysate's electrolytes, and perform other steps prior to treatment.

During treatment, the controller 627 may recirculate the dialysate, monitor the performance of the dialysate, maintain and adjust the pressure of the dialysate in the dialysis filter 602 and the dialysate filter 616, as well as manage any ongoing air removal from the dialysate and manage the electrolyte levels of the dialysate. During teardown, the controller 627 may remove some, much, or all of the dialysate using the fluid removal pump 640, reset many of the processing parameters, prepare the system to remove and replace tubing and the dialysis filter 602, dialysate filters 616, carbon filter 628, ultrafilter 630, and perform other functions to prepare the system for treating another patient.

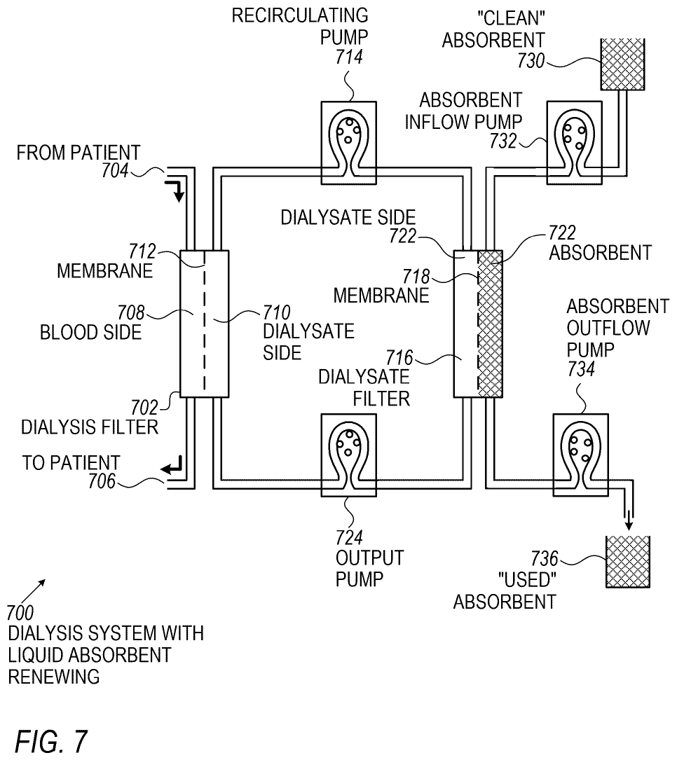

FIG. 7 is a dialysis system with liquid dialysate absorbent. Embodiment 700 is a simplified version of a dialysis system with recirculating dialysate, where an absorbent used to clean and rejuvenate or recharge the dialysate is a liquid absorbent.

Embodiment 700 shows a simplified diagram of a dialysis system that has a dialysis filter 702 through which a fluid, such as blood, comes from a patient 704 and exits after treatment at 706. The dialysis filter 702 may have a blood side 708 and a dialysate side 710, separated by a membrane 712.

The dialysate side may have a liquid dialysate that may be recirculated using a recirculating pump 714 through a dialysate filter 716. The dialysate filter 716 may have a membrane 718 which may separate the dialysate side 720 from an absorbent side 722. An outflow pump 724 may assist in recirculating the dialysate as well as monitoring and controlling dialysate pressure within the dialysis filter 702 and dialysate filter 716.

The absorbent 722 may be a liquid absorbent. A reservoir 730 of ‘clean’ absorbent may be connected to the dialysate filter 716 with an absorbent inflow pump 732. An absorbent outflow pump 734 may be used with the absorbent inflow pump 732 to fill the absorbent into the dialysate filter 716, control the pressure of the absorbent in the dialysate filter 716, and remove used absorbent to deposit in a collection reservoir 736.

When a liquid absorbent may be used in such a manner, absorbent may be periodically or continuously added and removed from the dialysate filter 716 during a dialysis treatment. In some use cases, the dialysate filter 716 may be initially filled with fresh absorbent, used to clean dialysate for a period of time, then flushed and replaced with fresh absorbent. In other use cases, the absorbent may be slowly removed and replaced during the dialysis treatment.

The foregoing description of the subject matter has been presented for purposes of illustration and description. It is not intended to be exhaustive or to limit the subject matter to the precise form disclosed, and other modifications and variations may be possible in light of the above teachings. The embodiment was chosen and described in order to best explain the principles of the invention and its practical application to thereby enable others skilled in the art to best utilize the invention in various embodiments and various modifications as are suited to the particular use contemplated. It is intended that the appended claims be construed to include other alternative embodiments except insofar as limited by the prior art.

1. A filter cartridge for filtering dialysate used in blood dialysis, said filter cartridge comprising:
a housing comprising a semi-permeable filter membrane;
said semi-permeable filter membrane having a dialysate side and a sink/source material side;
said dialysate side having an inlet through which dialysate passes into said filter cartridge in an unfiltered state, in said unfiltered state, said dialysate comprising a first set of components;
said dialysate side having an outlet through which said dialysate passes out from said housing in a filtered state;
when said filter cartridge is in a preliminary state, said sink/source material being configured to generate a first predetermined amount of said dialysate when a second predetermined amount of saline solution is passed through said filter cartridge;
after said generating said dialysate, said filter cartridge being in an operational state;
when in said operational state, said sink/source material configured to remove said first set of components from said dialysate to convert said dialysate from said unfiltered state to said filtered state.

2. The filter cartridge of claim 1, said first set of components comprising at least one of a group composed of:
urea;
potassium;
sodium;
phosphorous.

3. The filter cartridge of claim 1, said sink/source material comprising at least one of a group composed of:
gelatin;
agar;
pectin;
psyllium polysaccharide;
polystyrene sulfonate;
poly(sodium styrene sulfonate).

4. The filter cartridge of claim 1, said sink/source material having a molecular weight greater than 63,000.

5. The filter cartridge of claim 4, said sink/source material comprising calcium chloride and magnesium chloride.

6. The filter cartridge of claim 5, said sink/source material comprising at least some of calcium chloride, magnesium chloride, sodium chloride, potassium chloride such that when said Saline solution of 0.9% sodium chloride is passed through said filter cartridge in said preliminary state, said dialysate is produced.

7. The filter cartridge of claim 6, said dialysate having a concentration of sodium ion is in the range of 120 mM and 160 mM.

8. The filter cartridge of claim 6, said dialysate having a concentration of potassium ion is in the range of 0-3 mM.

9. The filter cartridge of claim 6, said dialysate having a concentration of calcium ion is in the range of 1.0 mM to 2.5 mM.

10. The filter cartridge of claim 6, said dialysate having a concentration of magnesium ion is in the range of 0.5 mM to 1.25 mM.

11. The filter cartridge of claim 6, said dialysate having a concentration of bicarbonate ion is in the range of 30 mM to 50 mM.

12. The filter cartridge of claim 6, said dialysate having a concentration of acetate is in the range of 3 mM to 6 mM.

13. The filter cartridge of claim 6, said sink/source material comprising a pore volume of at least 75%.

14. The filter cartridge of claim 13, said sink/source material being at least partially cross linked.

15. The filter cartridge of claim 6, said filtered dialysate having an electrical conductivity between 12.0 mS and 16.0 mS.

16. The filter cartridge of claim 15, said semi-permeable filter membrane being a plurality of hollow fibers.

17. (canceled)

18. (canceled)

19. (canceled)

20. (canceled

22. (canceled)

23. (canceled)

24. (canceled

25. (canceled)

26. (canceled)

27. A dialysate processing system comprising:
a recirculating pump to recirculate dialysate during a dialysis treatment;
an output connection adapted to transfer dialysate to a dialysis filter, said dialysis filter having a blood/dialysate membrane with a blood side and a dialysate side;
an input connection adapted to transfer dialysate from said dialysis filter;
a dialysate filter cartridge comprising a dialysate filter membrane having a dialysate side and a sink/source material side;
said dialysate filter cartridge having a preliminary state configured to create said dialysate when saline solution is passed through said dialysate filter thereby converting said dialysate filter from said preliminary state to an operational state;
a saline reservoir comprising a first predetermined volume of saline solution;
a controller configured at least in part to perform a first method to create dialysate, said first method comprising:
causing said first predetermined volume of saline solution to pass through said dialysate filter cartridge when said dialysate filter is in said preliminary state to create a second predetermined volume of said dialysate;
said controller configured at least in part to perform a second method to perform blood filtering, said second method comprising:
when said dialysate filter is in said operational state, activating said recirculating pump thereby causing said dialysate to recirculate through said dialysis filter, thereby changing said dialysate from a filtered state to an unfiltered state;
causing said dialysate to recirculate through said dialysate filter, thereby changing said dialysate from an unfiltered state to a filtered state.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

43. A method of performing dialysis, said method comprising:
introducing a first predetermined amount of saline solution into a dialysate filter being in a preliminary state to create a second predetermined amount of dialysate, after which, said dialysate filter being in an operational state;
when said dialysate filter is in said operational state, recirculating said dialysate between a dialysis filter and said dialysate filter, said dialysis filter receiving blood to be processed and discharging filtered blood and receiving filtered dialysate and discharging unfiltered dialysate;
said dialysate filter receiving said unfiltered dialysate and discharging said filtered dialysate;
said dialysate filter comprising sink/source material configured to extract a first set of components from said dialysate, said first set of components being extracted from said blood into said dialysate and being extracted from said unfiltered dialysate into a protein-based material.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)
